(19) United States
(12) Patent Application Publication
(10) Pub. No.: US 2021/0060609 A1
(43) Pub. Date: Mar. 4, 2021

(54) ULTRASONIC GENERATOR AND CONTROLLER FOR ULTRASONIC GENERATOR

(71) Applicant: Cybersonics, Inc., Erie, PA (US)

(72) Inventor: Shane A. Rock, McKean, PA (US)

(21) Appl. No.: 17/005,827

(22) Filed: Aug. 28, 2020

Related U.S. Application Data
(60) Provisional application No. 62/893,997, filed on Aug. 30, 2019.

(51) Int. Cl. B06B 1/02

(52) U.S. Cl. CPC B06B 1/0284; B06B 2201/76; B06B 2201/20

(57) ABSTRACT

An ultrasonic generator for a transducer is provided. The ultrasonic generator includes a function generator and a controller. The function generator is in signal communication with the transducer. The controller is in signal communication with the function generator and cooperates with the function generator to facilitate generation of a drive signal from the function generator to the transducer. An amplifier module is in signal communication with the function generator to amplify the drive signal.

### REFERENCE TO RELATED APPLICATION

This application claims priority of U.S. provisional patent application Ser. No. 62/893,997, entitled Ultrasonic Generator and Controller for Ultrasonic Generator, filed Aug. 30, 2019, and hereby incorporates this provisional patent application by reference herein in its entirety.

### TECHNICAL FIELD

The apparatuses and methods described below relate to an ultrasonic generator for operating a pair of transducers alternatively but in a manner that causes the pair of transducers to appear to be operating simultaneously.

### BACKGROUND

Conventional ultrasonic generators typically have multiple power boards for powering a pair of transducers and are therefore bulky, heavy, and expensive.

### BRIEF DESCRIPTION OF THE DRAWINGS

The following detailed description of embodiments can be best understood when read in conjunction with the drawings enclosed herewith:

FIG. 1 is a schematic view depicting an ultrasonic system, in accordance with one embodiment;

FIG. 2 is a schematic view depicting a switching circuit of the ultrasonic system of FIG. 1;

FIG. 3 is a plot depicting first and second output signals that are generated by the switching circuit of FIG. 2, in accordance with one embodiment; and

FIG. 4 is a schematic view depicting an ultrasonic system, in accordance with another embodiment.

### DETAILED DESCRIPTION

The various embodiments described below are generally directed to ultrasonic generators that facilitate operation of a transducer for use in any of a variety of medical applications apply ultrasonic energy to tissues or internal organs, such as a heart or a kidney, for example. The structures and techniques described often employ cyclical mechanical pressure energy, most often in the form of non-ablative low frequency ultrasonic energy. The energy may be generated at energy levels that are low enough to prevent significant heating of the tissues but high enough to penetrate into target tissues at levels that induce a desired level of shear stress. The energy levels can accordingly deliver therapeutic energy through a patient's body to provide therapeutic benefits without imposing significant trauma.

Ultrasonic energy can be understood to be a cyclic sound pressure that is applied to a patient's body at a frequency greater than about 20 kHz (e.g., the upper limit of human hearing) as part of a treatment or imaging regimen. The devices, products, and methods described herein may be employed to facilitate application of such ultrasonic energy to a patient's body. Certain embodiments may be particularly well suited for treatment of diseases that include an ischemic component, including coronary artery disease, occlusive diseases of the peripheral vasculature, erectile dysfunction, hypertension, diabetes, and the like. The exemplary embodiments may have their most immediate application for treatment of the kidneys or the heart. Such embodiments may ameliorate, mitigate, and/or avoid some or all acute or long term injury to tissues of the kidneys or the heart. Many of the embodiments may be described herein with reference to inhibiting injury to the kidneys associated with administration of contrast imaging agents, prior to and/or in conjunction with dialysis treatment, so as to inhibit progression of chronic kidney disease. Nonetheless, the structures and techniques described for these indications will often be suitable for additional therapies as can be understood with reference to the disclosure herein.

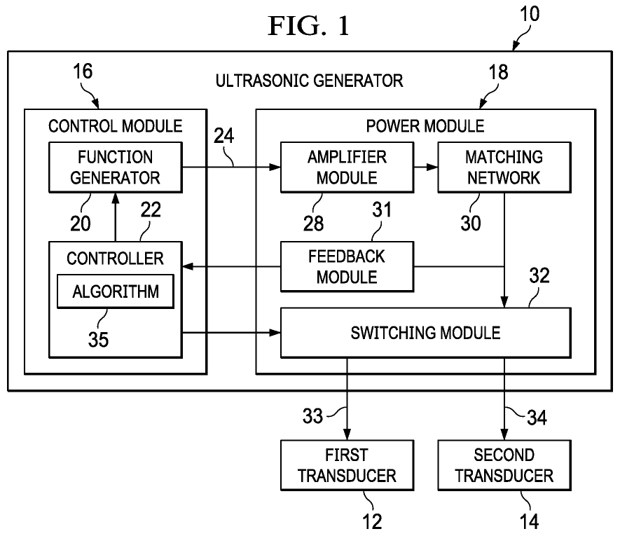

An ultrasonic generator 10 is illustrated in FIG. 1 to facilitate alternating operation of a first transducer 12 and a second transducer 14 in a manner that causes the first and second transducers 12, 14 to appear to be operating simultaneously. The first and second transducers 12, 14 can be placed together on a body of a patient and configured to supply ultrasonic (e.g., ultrasonic) energy (e.g., non-ablative energy) towards the patient to facilitate treatment and/or imaging. In one embodiment, the first and second transducers 12, 14 can include a fluid-filled chamber (not shown) with a flexible membrane (not shown) that serves as a patient interface, which can be positioned relative to the patient so as promote coupling of the first and second transducers 12, 14 with the skin of the patient. The patient interface can generally provide vibrational coupling of the skin and other tissues of the patient with pressure-signal transmitting surfaces of the first and second transducers 12, 14. One example of a suitable transducer is illustrated and described in U.S. patent application Ser. No. 16/993,911 which is hereby incorporated by reference herein in its entirety.

Still referring to FIG. 1, the ultrasonic generator 10 can include a control module 16 and a power module 18. In one embodiment, the control module 16 and the power module 18 can be provided as separate circuit boards. In another embodiment, the control module 16 and the power module 18 can be integrated onto a single circuit board. The control module 16 can include a function generator 20 and a controller 22 that is in signal communication with the function generator 20. The controller 22 can cooperate with the function generator 20 to facilitate generation of a drive signal 24 that facilitates control of the first and second transducers 12, 14, as will be described in further detail below.

The power module 18 can include an amplifier module 28, a matching network 30, a feedback module 31, and a switching module 32. The amplifier module 28 can be in signal communication with each of the function generator 20 and the matching network 30. The matching network 30 can be in signal communication with each of the feedback module 31 and the switching module 32. The switching module 32 can be in signal communication with each of the first and second transducers 12, 14 via respective first and second outputs 33, 34. The drive signal 24 can be transmitted from the function generator 20 and to the amplifier module 28 which can amplify the drive signal 24 to provide the desired output level. The resulting amplified drive signal can be transmitted to the matching network 30 which can be configured to transform (e.g., filter) the amplified drive signal into a transformed drive signal that is suitable to power the first and second transducers 12, 14 (e.g., a sinusoidal or square-wave waveform).

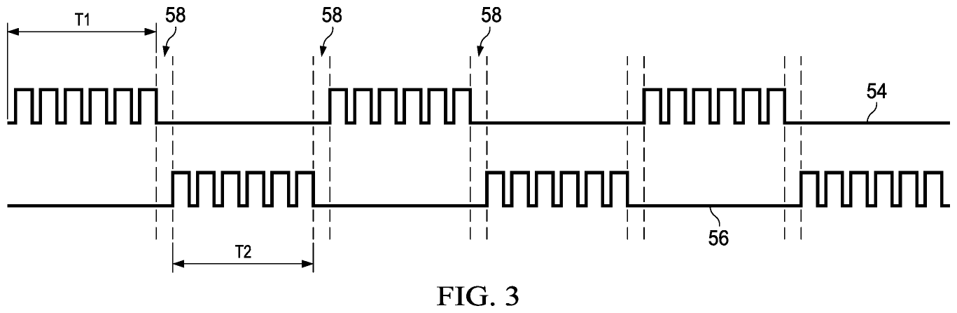

The transformed drive signal can be transmitted to the switching module 32. The switching module 32 can be configured to selectively route the transformed drive signal to either of the first and second outputs 33, 34 such that the transformed drive signal is only present on one of the first output 33 and the second output 34 at any given time. Such routing of the transformed drive signal between the first and second outputs 33, 34 can produce first and second output signals (e.g., 54, 56 in FIG. 3) that are out of phase with each other and thus activate the first transducer 12 and the second transducer 14 at different times, as will be described in further detail below. The controller 22 can be in signal communication with the switching module 32 and can be configured to facilitate control of the switching module 32 to control the routing of the transformed drive signal to either the first transducer 12 or the second transducer 14.

When the transformed drive signal is routed to either of the first and the second transducers 12, 14, the overall performance of the first and second transducer 12, 14 can be affected by various input parameters of the transformed drive signal, such as, for example, a voltage, a current, a frequency, or a duty cycle. The controller 22 can accordingly be configured to selectively vary at least one of the input parameters of the transformed drive signal (via the drive signal 24) to generate first and second output signals that facilitate individualized control of the operation of the first and second transducers 12, 14. In one embodiment, the controller 22 can vary the frequency of the transformed drive signal to generate first and second output signals that have respective frequencies that substantially match the resonant frequencies of the first and second transducers 12, 14. In situations where the first transducer 12 and the second transducer 14 have different resonant frequencies, the controller 22 can vary the frequency of the first and second output signals between two different frequencies depending upon whether the transformed drive signal is present on the first output 33 or the second output 34. For example, when the transformed drive signal is present on the first output 33, the controller 22 can control the frequency of the transformed drive signal to generate a first output signal that has a first frequency that matches the resonant frequency of the first transducer 12. However, when the transformed drive signal is present on the second output 34, the controller 22 can control the frequency of the transformed drive signal to generate a second output signal that has a second resonant frequency that matches the resonant frequency of the second transducer 14 different from the first resonant frequency.

The different resonant frequencies of the first and second transducers 12, 14 can be stored in the controller 22 (e.g., in memory) and cross-referenced to facilitate generation of the correct resonant frequency for the first and second transducers 12, 14. In one embodiment, the controller 22 can interrogate the first and second transducers 12, 14 to determine the resonant frequencies of the first and second transducers 12, 14 by first conducting a frequency sweep of the first and second transducers 12, 14 and then operating the first and second transducers 12, 14 at one or more of a minimum impedance, a maximum current, or a desired power factor.

The controller 22 can be configured to maintain the input parameters of the transformed drive signal within certain operational limits of the first and second transducers 12, 14, such as, for example, an input voltage range, an input current range, or an input power range. These operational limits can be stored in the controller 22 for cross-referencing during activation of the first and second transducers 12, 14. In one embodiment, the controller 22 can interrogate the first and second transducers 12, 14 to determine their operational limits.

Still referring to FIG. 1, the controller 22 can include an algorithm 35 that maintains the first and second transducers 12, 14 at a predefined operating condition, such as, for example, a minimum impedance, a maximum current, or a predefined power factor (e.g., a desired power factor). The controller 22 can be in communication with the feedback controller 31 such that the feedback controller 31 can provide feedback data to the algorithm 35. The feedback data can include relevant information about of the input parameters of the transformed drive signal (e.g., voltage, current, phase). The controller 22 can utilized the algorithm 35 to monitor at least one input parameter of the transformed drive signal, and make any adjustments, if necessary, to maintain the first and second transducers 12, 14 at the predefined operating condition defined by the algorithm 35. In one embodiment, the algorithm 35 can facilitate continuous adjustment of the frequency of the drive signal 24 to maintain the first and second transducers 12, 14 at a predefined operating condition.

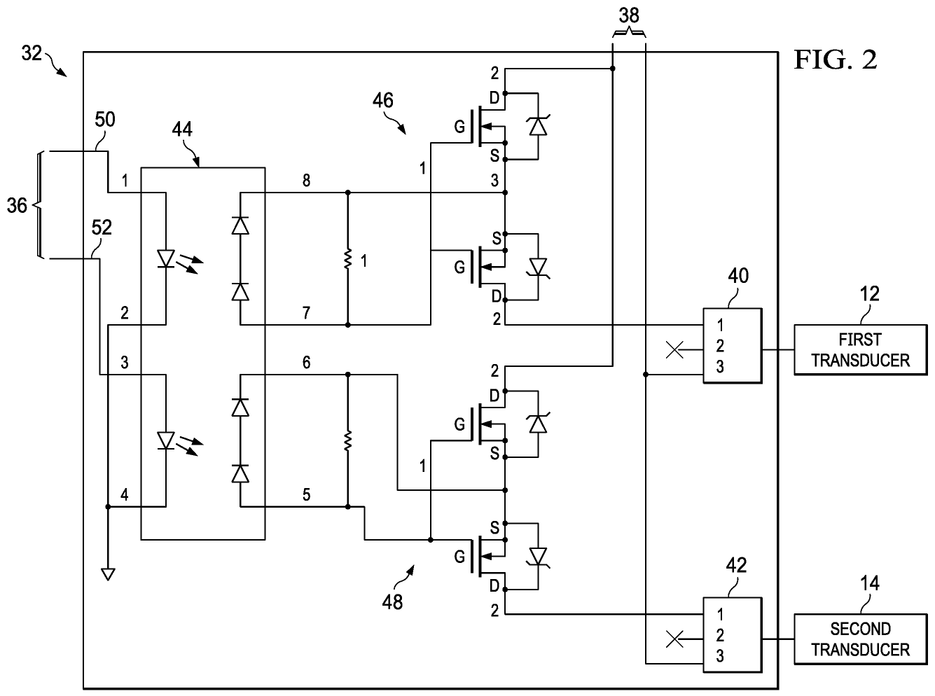

Referring now to FIG. 2, the switching module 32 can include a communication input port 36, a transformed drive signal input port 38, a first output port 40, and a second output port 42. The communication input port 36 can be coupled with an optocoupler 44 that is electrically coupled with a first switching circuit 46 and a second switching circuit 48. The transformed drive signal input port 38 can be coupled with the matching network 30 of FIG. 1 for receiving the transformed drive signal. The transformed drive signal input port 38 can be selectively, electrically coupled with the first output port 40 and the second output port 42 via the first switching circuit 46 and the second switching circuit 48, respectively. The first output port 40 and the second output port 42 can be electrically coupled with the first transducer 12 and the second transducer 14, respectively, to facilitate powering of the first transducer 12 and the second transducer 14 with the transformed drive signal.

The communication input port 36 can include a first enable line 50 and a second enable line 52 that are electrically coupled with the controller 22. The controller 22 can selectively and alternatively activate either the first enable line 50 or the second enable line 52 to facilitate routing of the transformed drive signal at the transformed drive signal input port 38 to the first output port 40 or the second output port 42, respectively. The optocoupler 44 can communicate the activation signal to the first and second switching networks 46, 48 while providing electrical isolation between the first and second enable lines 50, 52 and the first and second switching circuits 46, 48 to prevent the transformed drive signal from being inadvertently backfed to the controller 22. When the first enable line 50 is activated, the first switching circuit 46 can be activated (e.g., closed) and the transformed drive signal can be routed from the transformed drive signal input port 38, through the first switching circuit 46, to the first output port 40 and to the first transducer 12. When the second enable line 52 is activated, the second switching circuit 48 can be activated (e.g., closed) and the transformed drive signal can be routed from the transformed drive signal input port 38, through the second switching circuit 48, to the second output port 42, and to the second transducer 14.

During transmission of the transformed drive signal through the switching module 32, the controller 22 can alternate activation of the first enable line 50 and the second enable line 52 to alternate routing of the transformed drive signal between the first output port 40 and the second output port 42, respectively. In doing so, the transformed drive signal is cycled between the first and second transducers 12, 14 to alternatively drive the first and second transducers 12, 14 such that the first and second transducers 12, 14 are perceived to be operating simultaneously. FIG. 3 illustrates one example of first and second output signals 54, 56 that are produced as a function of alternating the routing of the transformed drive signal between the first output port 40 and the second output port 42 (e.g., via alternative activation of the first and second enable lines 50, 52). The first and second output signals 54, 56 can effectively be out of phase from one another such that the transformed drive signal is only present on one of the first and second output ports 40, 42 at any given time.

The period of time that each of the first and second output signals 54, 56 are present on each of the first and second output ports 40, 42, respectively (e.g., the duty cycle), the frequency of the first and second output signals 54, 56 (e.g., the modulation frequency), as well as other signal characteristics, can be controlled by the controller 22 to produce a desired output from the first and second transducers 12, 14 and/or to be compatible with the input power requirements of each of the first and second transducers 12, 14. The duty cycle can be between about 1% and about 100%, where any duty cycle above 50% will cause the first and second transducers to operate for unequal amounts of time (i.e. first transducer 12 operating for 60%, will cause the second transducer 14 to operate for 40%), and a duty cycle of 100% will allow only one of the transducers to operate. In one embodiment, the modulated frequency can be between about 1 Hz and about 100 Hz or more specifically between about 1 Hz and 25 Hz. In some embodiments, the modulated frequency can be varied during operation. Although the first and second output signals 54, 56 are shown in FIG. 3 to have substantially the same characteristics (e.g., duty cycle and modulated frequency) when present on either of the first and second output ports 40, 42, it is to be appreciated that, the duty cycle and/or the modulated frequency of the first and second output signals 54, 56 can be different to accommodate for different transducers and/or to achieve different operating conditions between the first and second transducers 12, 14.

In one embodiment, as illustrated in FIG. 3, when either of the first enable line 50 or the second enable line 52 is deactivated (e.g., to terminate transmission of the transformed drive signal on the first output port 40 and the second output port 42), the controller 22 can delay activation of the other enable line for a short period of time (e.g., dead time 58 where the transformed drive signal is not present on either the first or second output port 40, 42) to allow the controller 22 to tailor the transformed drive signal appropriately for the upcoming transducer before transmitting it to the that transducer.

The method for generating the first and second output signals 54, 56 illustrated in FIG. 3 will now be discussed. First, during transmission of the drive signal 24, the controller 22 can activate the first enable line 50 and can deactivate the second enable line 52 for a first time period T1 such that the transformed drive signal is present on the first output port 40. After the first time period T1 has elapsed, the first and second enable lines 50, 52 can be deactivated for a second time period (e.g., the dead time 58). Once the dead time 58 has elapsed, the second enable line 52 can be activated and the first enable line 50 can be deactivated for a second time period T2 such that the transformed drive signal is present on the second output port 42. After the second time period T2 has elapsed, the controller 22 can continuously repeat the above steps to generate the first and second output signals 54, 56 illustrated in FIG. 3. It is to be appreciated that, although the first and second output signals 54, 56 are shown to be square wave waveforms, the first and second output signals 54, 56 can be any of a variety of suitable waveforms for powering the first and second transducers 12, 14, such as sinusoidal, for example.

It is to be appreciated that alternating the routing of the transformed drive signal between the first output port 40 and the second output port 42 can facilitate alternative operation of two transducers (e.g., the first and second transducers 12, 14) with a single drive signal from a single function generator rapidly enough to cause the pair of transducers to appear to be operating simultaneously. As such, the ultrasonic generator 10 can be more compact and cost effective than certain conventional generators that require separate function generators, power amplifiers, and/or matching networks for each transducer that is being powered.

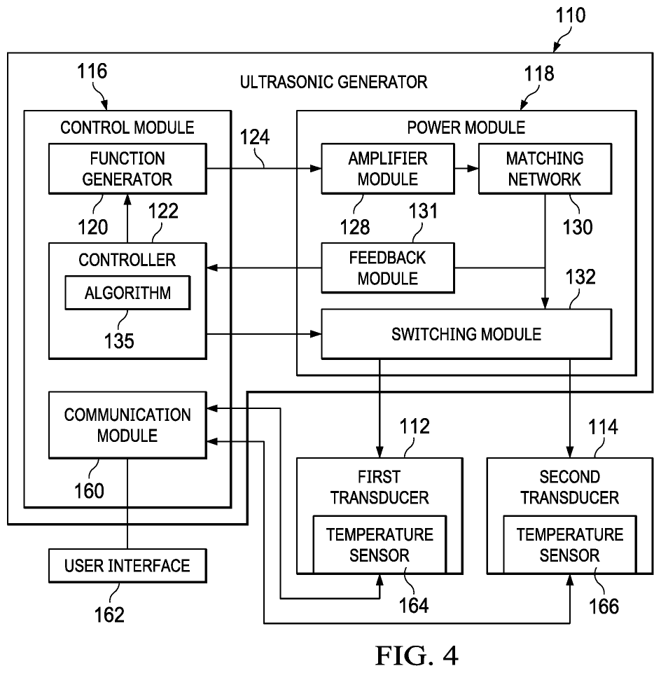

FIG. 4 illustrates an alternative embodiment of an ultrasonic generator 110 that is similar to, or the same as in many respects as, the ultrasonic generator 10 illustrated in FIG. 1. For example, the ultrasonic generator 110 can include a control module 116 and a power module 118. The control module 116 can include a function generator 120 and a controller 122 that cooperate to facilitate generation of a drive signal 124 controlled by an algorithm 135. The power module 118 can include an amplifier module 128, a matching network 130, a feedback module 131, and a switching module 132. The switching module 132 can be in signal communication with first and second transducers 112, 114. The drive signal 124 can be transmitted from the function generator 120 and to the amplifier module 128 which can amplify the drive signal 124 to compensate for any degradation of the drive signal. The amplified drive signal can be transmitted to the matching network 130, which can transform the drive signal into a waveform that is most appropriate to power the first and second transducers 112, 114 (e.g., a sinusoidal or square-wave waveform). The transformed drive signal from the matching network 130 can be transmitted to the switching module 132 for presentation to the first and second transducers 112, 114. The feedback module 131 can provide information about an electrical variable of the transformed drive signal to an algorithm 135.

The ultrasonic generator 110, however, can include a communication module 160 that is in signal communication (e.g., communicatively coupled) with the first and second transducers 112, 114 to obtain operational data therefrom. In one embodiment, the communication module 160 can be in wired communication with the first and second transducers 112, 114 (via a communication cable). In another embodiment, the communication module 160 can be in wireless communication with the first and second transducers 112, 114 via any of a variety of wireless communication protocols such as, for example, Wi-Fi, Cellular, or Wireless Personal Area Networks (WPAN) (e.g., IrDA, Bluetooth, Bluetooth Low Energy, Zigbee, wireless USB). Data obtained from the first and second transducers 112, 114 can be provided to a user via a user interface 162 that is in signal communication with a communication module 160. The user interface 162 can include a display (not shown) that allows a user to view the data gathered from the first and second transducers 112, 114.

The controller 122 can cooperate with the communication module 160 to facilitate interrogation of the first and second transducers 112, 114 prior to operation of the first and second transducers 112, 114 to determine the resonance frequencies, the operational limits, or other relevant information about the first and second transducers 112, 114. In one embodiment, the controller 122 can interrogate the first and second transducers 112, 114 to confirm that the first and second transducers 112, 114 are compatible with the particular treatment or imaging regimen prescribed to a patient. In such an embodiment, the first and second transducers 112, 114 can be assigned unique identifying information, such as a model number, a unique address, or a unique serial number. When the first and second transducers 112, 114 are communicatively coupled with the communication module 160, the controller 122 can identify the first and second transducers 112, 114 based upon their identifying information and can prevent operation of the ultrasonic generator 110 if the first and second transducers 112, 114 are not compatible with the particular treatment or imaging regimen that is being prescribed to the patient.

Still referring to FIG. 4, the first and second transducers 112, 114 can each include a temperature sensor 164, 166 respectively, that is in signal communication with the communication module 160 such that the communication module 160 can gather temperature data from the temperature sensors 164, 166 to facilitate detection of the temperature of the first and second transducers 112, 114. During operation of ultrasonic generator 110, the temperature of the first and second transducers 112, 114 can be displayed on the user interface 162. If the first transducer 112 and/or the second transducer 114 overheats (e.g., exceeds a threshold temperature), such as, for example, when a cooling fluid system becomes blocked, the user interface 162 can issue an alarm to notify the user. In one embodiment, the ultrasonic generator 110 can additionally or alternatively be automatically shut off to allow the overheating condition to be corrected.

The foregoing description of embodiments and examples has been presented for purposes of illustration and description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent devices by those of ordinary skill in the art. Rather, it is hereby intended that the scope be defined by the claims appended hereto. Also, for any methods claimed and/or described, regardless of whether the method is described in conjunction with a flow diagram, it should be understood that unless otherwise specified or required by context, any explicit or implicit ordering of steps performed in the execution of a method does not imply that those steps must be performed in the order presented and may be performed in a different order or in parallel.

What is claimed is:

1. An ultrasonic generator comprising:
a control module comprising a function generator and a controller in signal communication with the function generator and cooperating with the function generator to facilitate generation of a drive signal from the function generator;
a power module comprising:
an amplifier module in signal communication with the function generator and configured to amplify the drive signal into an amplified drive signal;
a matching network in signal communication with the amplifier module and cooperating with the amplifier module to transform the amplified drive signal into a transformed drive signal that is compatible with a first transducer and a second transducer; and
a switching module in signal communication with the controller and comprising a first output and a second output, the switching module being configured to selectively route the transformed drive signal between the first output or the second output to facilitate independent activation of a first transducer and a second transducer, wherein the controller is operably coupled with the switching module and is configured to facilitate control of the routing of the transformed drive signal between the first output and the second output.

2. The ultrasonic generator of claim 1 wherein the controller is configured to vary an input parameter of the transformed drive signal based upon whether the transformed drive signal is present on the first output or the second output.

3. The ultrasonic generator of claim 2 wherein the input parameter comprises a frequency and the controller is configured to vary the frequency of the transformed drive signal between a first frequency when the transformed drive signal is present on the first output and a second frequency when the transformed drive signal is present on the second output.

4. The ultrasonic generator of claim 3 wherein the first frequency comprises a resonant frequency of the first transducer and the second frequency comprises a resonant frequency of the second transducer.

5. The ultrasonic generator of claim 1 wherein routing the transformed drive signal between the first output and the second output facilitates production of a first output signal and a second output signal on the first output and the second output, respectively.

6. The ultrasonic generator of claim 5 wherein the first output signal is out of phase with the second output signal.

7. The ultrasonic generator of claim 6 wherein at least one of the first output signal and the second output signal has a modulation frequency of between about 1 Hz and about 25 Hz.

8. The ultrasonic generator of claim 6 wherein at least one of the first output signal and the second output signal has a duty cycle of between about 1% and 100%.

9. The ultrasonic generator of claim 1 wherein the control module and the power module are provided on individual circuit boards.

10. A system comprising:
a first transducer configured to supply ultrasonic energy towards a patient;
a second transducer configured to supply ultrasonic energy towards a patient;
an ultrasonic generator comprising:
a control module comprising a function generator and a controller in signal communication with the function generator and cooperating with the function generator to facilitate generation of a drive signal from the function generator;
a power module comprising:
an amplifier module in signal communication with the function generator and configured to amplify the drive signal into an amplified drive signal;
a matching network in signal communication with the amplifier module and cooperating with the amplifier module to transform the amplified drive signal into a transformed drive signal that is compatible with a first transducer and a second transducer; and
a switching module in signal communication with the controller and comprising a first output in signal communication with the first transducer and a second output in signal communication with the second transducer, the switching module being configured to selectively route the transformed drive signal between the first output and the second output to facilitate independent activation of the first transducer and the second transducer, wherein the controller is operably coupled with the switching module and is configured to facilitate control of the routing of the transformed drive signal between the first output and the second output.

12. The system of claim 11 wherein the controller is configured to vary an input parameter of the transformed drive signal based upon whether the transformed drive signal is present on the first output or the second output.

13. The system of claim 12 wherein the input parameter comprises a frequency and the controller is configured to vary the frequency of the transformed drive signal between a first frequency when the transformed drive signal is present on the first output and a second frequency when the transformed drive signal is present on the second output.

14. The system of claim 13 wherein the first frequency comprises a resonant frequency of the first transducer and the second frequency comprises a resonant frequency of the second transducer.

15. The system of claim 14 wherein the controller is further configured to interrogate the at least one of the first transducer and the second transducer to determine the resonant frequency of the at least one of the first transducer and the second transducer.

16. The system of claim 15 wherein the controller is further configured to interrogate the at least one of the first transducer and the second transducer by conducting a frequency sweep of the at least one of the first transducer and the second transducer.

17. The system of claim 11 wherein routing the transformed drive signal between the first output and the second output facilitates production of a first output signal and a second output signal on the first output and the second output, respectively.

18. The system of claim 17 wherein the first output signal is out of phase with the second output signal.

19. The system of claim 11 further comprising an algorithm that maintains at least one of the first transducer and the second transducer at a predefined operating condition.

20. The system of claim 19 wherein the predefined operating condition comprises one of a minimum impedance, a maximum current, and a power factor.

21. The system of claim 20 wherein the algorithm can facilitate continuous adjustment of a frequency of the transformed drive signal to maintain at least one of the first transducer and the second transducer at the predefined operating condition.

22. A system comprising:
a transducer configured to supply ultrasonic energy towards a patient;
an ultrasonic generator comprising:
a function generator in signal communication with the transducer;
a controller in signal communication with the function generator and cooperating with the function generator to facilitate generation of a drive signal;
an amplifier module in signal communication with the function generator and configured to amplify the drive signal; and
a communication module in signal communication with the transducer and configured to cooperate with the controller to facilitate interrogation of the transducer to obtain operational data therefrom.

23. The system of claim 22 wherein the operational data comprises identifying information and the controller is configured to prevent operation of the transducer based upon the identifying information.

24. The system of claim 22 wherein the transducer further comprises a temperature sensor and the operational data comprises temperature data that facilitates detection of the temperature of the transducer.

25. The system of claim 24 wherein the controller is configured to prevent operation of the transducer based upon the temperature data.

26. The system of claim 22 further comprising a matching network in signal communication with the amplifier module and the transducer, the matching network cooperating with the amplifier module to transform the drive signal into a transformed drive signal for transmission to the transducer.
